(19) United States
(12) Patent Application Publication
(10) Pub. No.: US 2026/0224807 A1
(43) Pub. Date: Aug. 6, 2026

(54) PUMPLESS FLUID DELIVERY METHOD AND APPARATUS

(71) Applicant: Equilibar, LLC, Fletcher, NC (US)

(72) Inventors: RYAN MATTHEW HEFFNER, Fletcher, NC (US); CARLYLE LEONARD DONEVANT, IV, Fletcher, NC (US); JEFFREY DEAN JENNINGS, Fletcher, NC (US); KARL JOSEPH LUTKEWITTE, Fletcher, NC (US)

(21) Appl. No.: 19/149,739

(22) PCT Filed: Jan. 23, 2024
(86) PCT No.: PCT/US2024/012567

§ 371 Date: Jul. 21, 2025

Related U.S. Application Data
(60) Provisional application No. 63/440,683, filed on Jan. 23, 2023.

(51) Int. Cl. A61M 5/168; A61M 5/148

(52) U.S. Cl. CPC A61M 5/16804; A61M 5/1486; A61M 5/16886; A61M 2205/3334; A61M 2205/50

(57) ABSTRACT

A pumpless fluid delivery apparatus includes: a pressure housing having an outlet tube; and a closed-loop flow control apparatus coupled to the outlet tube, the flow control apparatus including a dome-loaded diaphragm control valve, an electronic pressure regulator, and an electronic flow controller operably coupled to the electronic pressure regulator and the control valve.

### BACKGROUND OF THE INVENTION

The present invention relates to fluid delivery methods and apparatus, and more particularly to pumpless fluid delivery.

The pharmaceutical industry has had long-standing requirements for flow generating devices to be accurate, cleanable, drainable, and easily maintained. To service this need, it is common to find positive displacement pumps as flow generating devices, since they can be designed with smooth transitions to accept surface polishing, as well as open fully to access internal surface areas to be cleaned in place. As these technologies grew in usefulness, the accuracy and performance benefits improved.

When the single use industry emerged as a new part of the pharmaceutical manufacturing space, the industry adopted the existing pharmaceutical pump designs to optimize for single-use manufacturing and use. Common designs for pumps include diaphragm pumps (quattroflow), or peristaltic pumps that pinch tubing to generate flow. These pumps work well for metering flow since their predictable swept volume can be useful for total volume filling applications. However, due to the single-use nature of the wetted elements, this results in degradation of the control volume over time, which can diminish this swept volume effect as the pump wears from use.

Many of the positive displacement pumps also suffer from a push through flow phenomenon, where higher inlet pressures (known to be 0.2 bar or greater) can push flow through the pump even when it's inoperable. This can reduce the flow range/effectiveness range of the pump, and affect particularly low flow requirements. Low flows are needed in many pharmaceutical processes, from gradient elution low dosing mixing for extremity buffer ranges, to small volume filling of single use bags. The quattroflow pump brand is an example of a pump that suffers from this type of low flow issue, but is commonly used in the industry.

Peristaltic pumps have a built-in check valve operation, but suffer from separate issues. These pumps use spring loaded rollers to pinch flexible tubing to generate flow. However, this action causes extreme pulsations, and also creates a type of mini-stagnation flow. As the roller disengages with the wall of compression, there's an isolated volume of tubing that is at much less pressure than the head pressure on the pump. As this new section of tubing is exposed to the flow path, there's a reverse pressure gradient, which causes a reverse in the direction of flow to back-fill this volume to get to pressure. This will generally be a small volume event, but proportional to the volume expansion of tubing and the RPM of the pump. In low RPM cases and large swept volume peristaltic pumps, this can cause fairly large changes in pressure. This can often outweigh the benefits that peristaltic pumps offer.

Finally, all positive displacement pumps suffer from the same issue; flow is interrupted for 50 % of the cycle of the pump due to the need to constantly re-charge the reservoir that's used to pump fluid. This creates pulsations in the flow profile, which can be damaging to sensitive cell media. Furthermore, the operating mode can be torturous for the fluid, which can impart high shear values. In a publicly available paper by Levitronix, a single use centrifugal pump company, it quantified shear values of its centrifugal pump vs PD pumps, showing that some are highly damaging to cells. Levitronix's technology can be limiting in other factors, requiring imparted energy through turbines at high RPMs, which can add heat.

In summary, all pumps convert added electrical energy through a motor to fluid power through generated motion. This transfer of energy can result in damage depending on the mechanism used (positive displacement or centrifugal). It would be highly preferred to have a flow generating system that avoided torturous fluid handling through pinching or high RPM rotation. Advanced medicines like gene therapy rely on complex long-chain proteins that can be irreversibly damaged. It's known in the industry that once damage to a protein chain occurs, it comes easier to damage further and further. Additionally, traditional pharma using biologics and cell walls can be damaged and lysed open prior to desired, which reduces yield and effectiveness of the process.

Accordingly, there is a need for a pumpless system.

### BRIEF SUMMARY OF THE INVENTION

This need is addressed by a pumpless fluid delivery apparatus including a source configured to supply a pressurized liquid, coupled to a closed-loop flow control apparatus including a dome-loaded diaphragm valve and an electronic flow controller.

According to one aspect of the technology described herein, a pumpless fluid delivery includes: a pressure housing having an outlet tube; and a closed-loop flow control apparatus coupled to the outlet tube, the flow control apparatus including a dome-loaded diaphragm control valve, an electronic pressure regulator, and an electronic flow controller operably coupled to the electronic pressure regulator and the control valve.

According to another aspect of the technology described herein, a pumpless fluid delivery method includes: providing a pressure housing having an outlet tube; filling the pressure housing with a liquid; admitting pressurized gas into the pressure housing; and metering a flow rate of the liquid using a closed-loop flow control apparatus coupled to the outlet tube, the flow control apparatus including a dome-loaded diaphragm control valve, an electronic pressure regulator, and an electronic flow controller operably coupled to the electronic pressure regulator and the control valve.

### BRIEF DESCRIPTION OF THE DRAWINGS

The invention may be best understood by reference to the following description taken in conjunction with the accompanying drawing figures in which:

FIG. 1 illustrates a pumpless fluid delivery apparatus;

FIG. 2 illustrates an optional control configuration for the apparatus of FIG. 1;

FIG. 3 is an enlarged view of a passthrough assembly shown in FIG. 1;

FIG. 4 shows the apparatus of FIG. 1, configured for use without a bag; and

FIG. 5 shows the apparatus of FIG. 1, configured for recirculating flow.

### DETAILED DESCRIPTION OF THE INVENTION

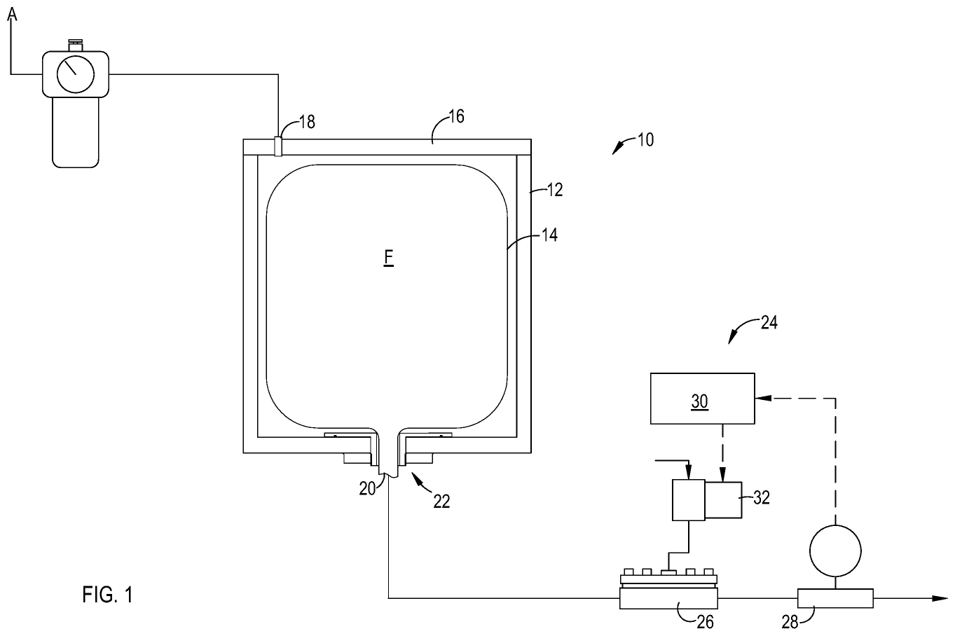

Referring to the drawings wherein identical reference numerals denote the same elements throughout the various views, FIG. 1 illustrates an exemplary embodiment of a pumpless fluid delivery apparatus 10.

A pressure housing 12, for example stainless steel, supports therein a single use bag 14 which is filled with process fluid “F”. A removable sealed lid 16 provides access for loading the bag 14.

A pressurization port 18 is disposed in the pressure housing 12. In use, it is coupled to a pressure source such as shop air “A”.

The bag 14 includes an outlet tube 20 which penetrates the pressure housing 12 via a passthrough assembly 22. The passthrough assembly 22 provides for leak proof sealing of the outlet tube 20 and the pressure housing 12. Example configurations are described in more detail below.

Downstream of the pressure housing 12, the outlet tube 20 connects to a flow metering apparatus 24 which utilizes a control valve 26 in the form of a dome-loaded direct sealing diaphragm valve arranged for closed loop feedback control. One example of a suitable valve is an SDO series valve from EQUILIBAR.

In the example of FIG. 1, the flow metering apparatus 24 includes a single use flow meter 28, which could be a wide variety of available Coriolis, ultrasonic, or other single use technology flow meters, a proportional-integral-derivative (PID) controller 30, and an electronic air pressure regulator 32. It is noted that items 30 and 32 can be combined to offer a more simple package, but can also work as discretely separate devices.

In operation, the control valve 26 would control against the pressurized value on the bag 14, where for a given pressure X, the control valve 26 would be greater than X to decrease/block flow, and less than X to increase or fully open for flow. Pressure X is set by the pressure reducing valve (not itemized) supplied by shop air “A”.

In this arrangement, the flow meter 28 functions to provide real-time updates to the PID controller 30, optionally with custom gain curves loaded. The flow meter 28 response may be in the range of about 10 ms to about 20 ms or faster. The control valve 26 is used to provide high resolution and high rangeability flow control.

The electronic air pressure regulator 32 may be controlled by input current (I/P) or voltage (E/P) signal and also includes a high speed PID loop adapted with value-adding logic such as custom gain curves and other programming for providing highly resolute flow control. An I/P or E/P device is an industry standard electro-pneumatic device that receives an electrical signal to control an incoming air supply to a desired output pressure. In a specific example, said flow control provides for a PID calculation action more than 800 times/second to stabilize the control valve 26 in the extreme ends of the flow ranges required. For example, some applications may require flow turndown ranges on the order of 100:1 to 1000:1.

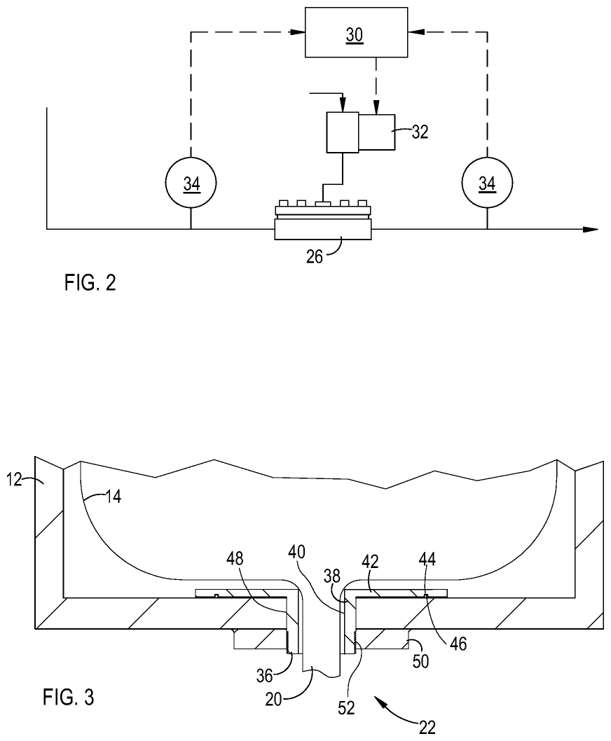

Alternatively, knowing the vessel pressure, upstream pressure, and downstream pressure, flow could be interpolated through accurate knowledge of the control valve 26 and its pressure vs flow curve. In this option, the flow meter would be replaced with a pair of pressure transducers 34. This is shown in FIG. 2.

The supplied pressure needs to be set to greater than system requirements. An example pressure would be 10 bar (4 bar peak pressure for single use systems). To get to that pressure, air is applied to the pressure housing 12, which would compress the single use bag 14 filled with liquid. Since liquid is incompressible, the bag 14 wouldn't suffer damage, and be nearly 0 pressure differential from the inside to outside (aside from hydrostatic head pressures, which would be minimal and a function of the height of the bag). This external pressure applied inside the pressure housing 12 but outside of the bag 14 will result in uniform even pressure applied. Since this application is targeted for valves filled only with liquids (which are generally incompressible), the bag 14 won't change volume significantly when pressurized, which will result in minimal stresses on the bag during the pumping operation. A big benefit of this method is it reduces stress on the polymer weld seams. Single use bags are created by welding polymer film together to constitute 2D or 3D shapes. Keeping the dP across the weld at 0 psid results in no shearing effect, which allows these weak pressure rated bags to survive to hundreds of psi.

The pressure housing 12 will need tubing penetrations to the external environment, which will have a differential pressure across it. Part of the advantage of this concept is that the pressure housing can withstand high differentials in applied pressure (typically 4-10 bar), as well as adapt to multiple bag and tubing sizes. Furthermore, in the spirit of single use, the system should be quickly set up to avoid cumbersome assembly details. Finally, the union of this joint needs to be aseptic, where the internals of the process remain in a closed volume until the system is ready to be run. That means that the single use portion cannot be assembled on-site to create the sterile boundary. These functions are accommodated by the passthrough assembly 22.

The purpose of this assembly 22 is to support expandable tubing to prevent rupturing, as well as allow the penetration to seal the internal air pressure applied to prevent significant leaks. Commonly, the tubing is penetrated through a pipe-like shaft. This pipe should have enough clearance to ensure that the tubing with hose barb or other aseptic connection can fit through, but not so large that the tubing must expand significantly to “inflate”, which would cause the hose to rupture. Typical embodiments would need to calculate the hoop stress of the tubing to ensure that the radius expansion does not exceed yield ratings of the tubing material.

This assembly 22 utilizes a “pressure inflated” geometry, where upon initial expansion of the tubing, the tubing compresses against the pipe walls. This inflation seals the leak path, and allows for the joint to remain unbroken during the operation of the cycle until liquid supply is exhausted.

A specific example embodiment is shown in FIG. 3. The passthrough assembly 22 includes a male component 36 of plastic or similar which includes a pipe like tube 38 with a central bore 40 and a flange 42 that includes a seal groove 44. The seal groove 44 retains an O-ring 46 in place for sealing between the male component 36 and the pressure housing 12. The central bore 40 is sized as described above relative to the outlet tube 20 to achieve a pressure seal, and the exterior of the tube 38 is sized to fit through an opening 48 in the pressure housing 12. A female nut 50 of plastic or similar is provided. The male component 36 and female nut 50 include complementary locking features 52, such as a locking/twisting action to create a breach type ¼ turn lock, or threaded lock like a parallel thread. This twisting action would engage the o ring to seal the pipe penetration, and allow for sealing. These components would slip onto the tubing to allow for a tight fit and minimal expansion to energize the sleeve seal. These plastic pieces would retain the o rings in place, and be ready-to-install for each single use consumption. They would support the bag from expanding out into the environment and rupturing. This male component 36 would be assembled on the outlet tubing 20 as an intermediate feature that would be done in the tube set assembly. The female nut 50 would slip over the end connection on the bag 14 to clamp the pressure housing 12 into place, creating an air-tight seal.

### Advantages

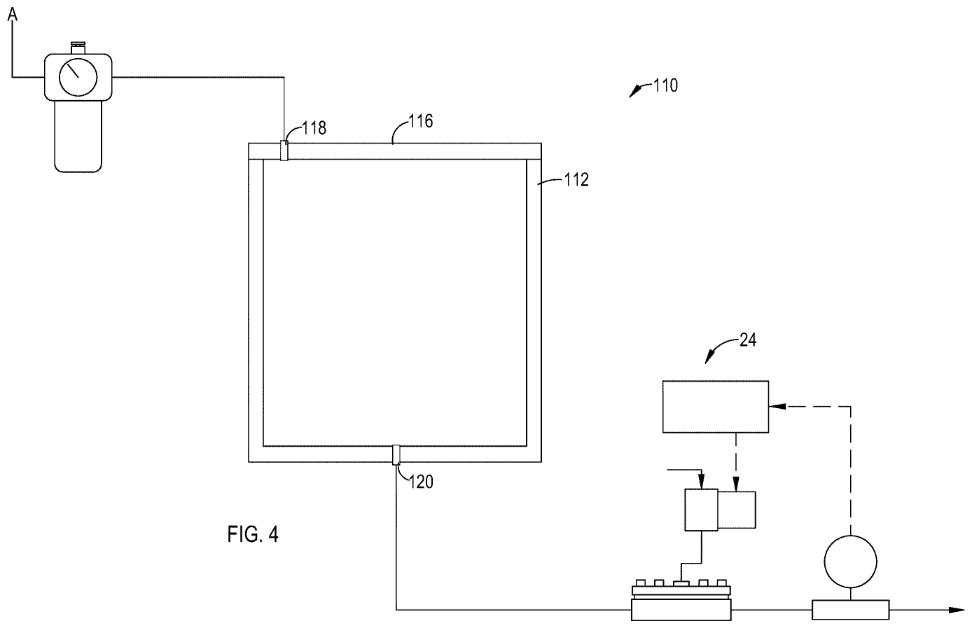

This apparatus and method solves the major challenges that pumps impose through their transfer from electric energy to fluid power, which creates turbulence, shear, heat, or pulsations (or any combination thereof). This could be used in any single use system, but the concepts can easily apply to multi-use systems. For example, FIG. 4 shows a pumpless fluid delivery apparatus 110 having a pressure housing 112 and a removable sealed lid 116 or other means for filling. A pressurization port 118 is disposed in the pressure housing 112. In use, it is coupled to a pressure source such as shop air “A”. An outlet tube 120 ais coupled to the pressure housing 112 Downstream of the pressure housing 112, the outlet tube 120 connects to a flow metering apparatus 24 which may be of the same type and functioning as described above.

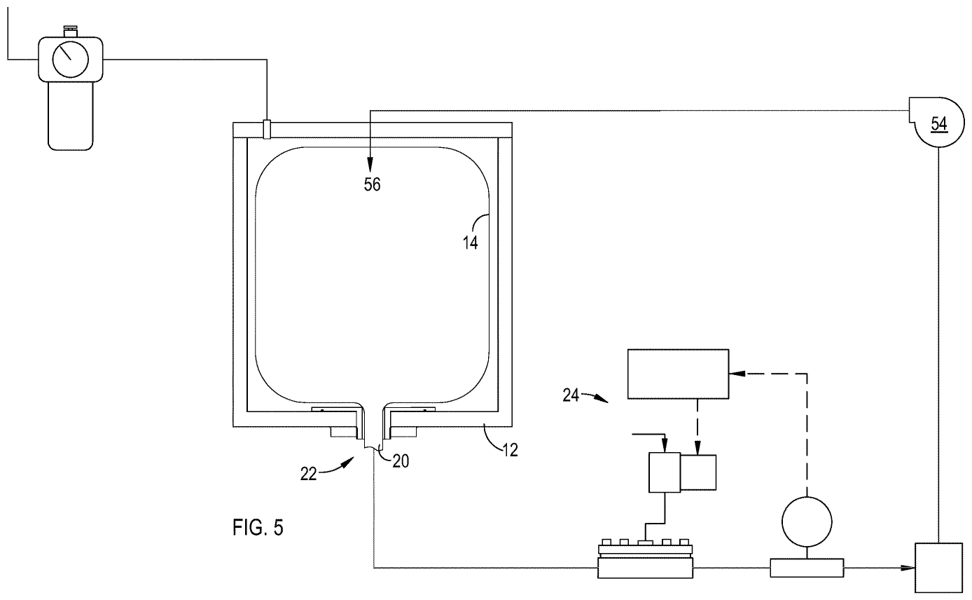

Finally, the system shown is largely representative of a batch process, but can be made continuous if a pump is added. Referring to FIG. 5, this could be achieved if the bag 14 included a return port 56, where an outlet of a recirculation pump 54 could be attached. Fluid recirculated in this way would benefit from this as opposed to just using a pump alone, since fluid returned to the bag 14 already underwent initial processing (example, chromatography, multi-pass TFF, etc.). This would result in an overall less damaging profile to the fluid since the fluid had the opportunity be processed first and then pumped.

The foregoing has described a pumpless fluid delivery apparatus and method. All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends any novel one, or any novel combination, of the features disclosed in this specification (including any accompanying claims, abstract and drawings), or to any novel one, or any novel combination, of the steps of any method or process so disclosed.

What is claimed is:

1. A pumpless fluid delivery apparatus, comprising:
a pressure housing having an outlet tube; and
a closed-loop flow control apparatus coupled to the outlet tube, the flow control apparatus including a dome-loaded diaphragm control valve, an electronic pressure regulator, and an electronic flow controller operably coupled to the electronic pressure regulator and the control valve.

2. The apparatus of claim 1, further comprising:
a flexible bag disposed in the pressure housing, the flexible bag including the outlet tube, wherein the outlet tube extends through a pass through assembly of the pressure housing; and
wherein the pressure housing is configured to admit pressurized gas between the flexible bag and a wall of the housing.

3. The apparatus of claim 2, wherein the pass through assembly includes a tube having a central bore which receives the outlet tube of the bag, the central bore sized so as to seal against the outlet tube by elastic expansion of the outlet tube at a predetermined pressure.

4. The apparatus of claim 3, wherein the pass through assembly includes:
a male component comprising the tube and a seal flange disposed inside the pressure housing; and
a female component disposed outside the pressure housing and connected to the male component by mutually engaged complementary locking features.

5. The apparatus of claim 1, wherein the closed-loop flow control apparatus includes a flow meter operably coupled to the electronic flow controller.

6. The apparatus of claim 1, wherein the closed-loop flow control apparatus includes a pressure transducer upstream of the control valve, and a pressure transducer downstream of the control valve.

7. A pumpless fluid delivery method, comprising:
providing a pressure housing having an outlet tube;
filling the pressure housing with a liquid;
admitting pressurized gas into the pressure housing; and
metering a flow rate of the liquid using a closed-loop flow control apparatus coupled to the outlet tube, the flow control apparatus including a dome-loaded diaphragm control valve, an electronic pressure regulator, and an electronic flow controller operably coupled to the electronic pressure regulator and the control valve.

8. The method of claim 7, wherein the liquid is contained in a flexible bag that is placed in the pressure housing, the flexible bag including the outlet tube which extends through a pass through assembly of the pressure housing; and wherein the gas is admitted between the bag and a wall of the pressure housing.

9. The method of claim 8, wherein the pass through assembly includes a tube having a central bore which receives the outlet tube of the bag, the central bore sized so as to seal against the outlet tube by elastic expansion of the outlet tube at a predetermined pressure.

10. The method of claim 9, wherein the pass through assembly includes:
a male component comprising the tube and a seal flange disposed inside the pressure housing; and
a female component disposed outside the pressure housing and connected to the male component by mutually engaged complementary locking features.

11. The method of claim 7, wherein the closed-loop flow control apparatus includes a flow meter operably coupled to the electronic flow controller.

12. The method of claim 7, wherein the closed-loop flow control apparatus includes a pressure transducer upstream of the control valve, and a pressure transducer downstream of the control valve.
